(19) United States
(12) Patent Application Publication
(10) Pub. No.: US 2026/0175049 A1
(43) Pub. Date: Jun. 25, 2026

(54) SHIELDING CHAMBERS FOR RADIATION PROTECTION, RADIATION PROTECTION SHIELDING DEVICES AND FLASH THERAPY SYSTEMS

(71) Applicant: MEVION MEDICAL EQUIPMENT CO., LTD., Suzhou (CN)

(72) Inventors: Jinliang ZHANG, Suzhou (CN); Zhihong ZHENG, Suzhou (CN); Jinwen SHUAI, Suzhou (CN)

(73) Assignee: MEVION MEDICAL EQUIPMENT CO., LTD., Suzhou (CN)

(21) Appl. No.: 19/537,481

(22) Filed: Feb. 11, 2026

Related U.S. Application Data
(63) Continuation of application No. PCT/CN2024/113328, filed on Aug. 20, 2024.

(30) Foreign Application Priority Data
Sep. 1, 2023 (CN) 202311122794.4

(51) Int. Cl. A61N 5/10; A61B 6/10

(52) U.S. Cl. CPC A61N 5/1049; A61B 6/107; A61N 5/1042

(57) ABSTRACT

Embodiments of the present disclosure provide a shielding chamber for radiation protection, a radiation protection shielding device, and a FLASH therapy system. The shielding chamber for radiation protection includes an accommodation cavity and a first window and a second window arranged sequentially at intervals along a direction of a beam on the shielding chamber. The accommodation cavity is located between the first window and the second window. The first window and the second window are respectively in communication with the accommodation cavity. The first window is used for entry of the beam. The second window is used for a target object to pass through. The target object is accommodated in the accommodation cavity after passing through the second window. A geometric center point of the accommodation cavity is set as a Bragg peak of the beam.

### CROSS-REFERENCE TO RELATED APPLICATIONS

This application is a continuation of International Application No. PCT/CN2024/113328, filed on Aug. 20, 2024, which claims priority of Chinese Patent Application No. CN202311122794.4, filed on Sep. 1, 2023, the contents of each of which are hereby incorporated by reference.

### TECHNICAL FIELD

The present disclosure relates to a field of proton accelerators and tumor radiotherapy, and in particular to a shielding chamber for radiation protection, a radiation protection shielding device, and a FLASH therapy system.

### BACKGROUND

Proton tumor therapy technology is a relatively advanced, mature and high-end medical technology internationally at present, and is one of the main means for tumor treatment. Compared with high-energy neutrons and electrons widely used at present, protons may concentrate the energy of rays more effectively on the tumor target region requiring treatment, and improve the local tumor control rate. Meanwhile, protons can greatly reduce the probability of radiation complications occurring in normal organs and tissues. Proton therapy devices have become the mainstream equipment for tumor radiotherapy internationally at present.

FLASH radiotherapy refers to that the radiation dose is delivered to the entire treatment volume in less than one second. Early preclinical evidence has shown that, compared with conventional radiotherapy, these extremely high dose rates may significantly protect healthy tissues without reducing the damage to cancer cells. Due to the fact that such FLASH high beam current delivers an enormous radiation dose in a very short time, extremely high requirements are put forward for radiation shielding walls. In other words, the site for performing FLASH radiotherapy shall have a sufficiently strong radiation shielding capacity. This requires the radiation shielding walls to be made sufficiently thick, which makes the walls occupy a large area and require a huge amount of capital investment.

Therefore, it is desired to provide a shielding chamber for radiation protection, a radiation protection shielding device and a FLASH therapy system, so as to achieve the radiation shielding effect and reduce the capital investment.

### SUMMARY

One or more embodiments of the present disclosure provide a shielding chamber for radiation protection. The shielding chamber for radiation protection includes an accommodation cavity and a first window and a second window arranged sequentially at intervals along a direction of a beam on the shielding chamber. The accommodation cavity is located between the first window and the second window. The first window and the second window are respectively in communication with the accommodation cavity. A center point of the first window, a center point of the second window, and a central axis of the beam are on the same straight line. The first window is used for entry of the beam. A size of the first window is smaller than a size of the second window. The second window is used for a target object to pass through. The target object is accommodated in the accommodation cavity after passing through the second window. The shielding chamber is located inside a treatment room. The second window is close to a radiation shielding wall of the treatment room. The radiation shielding wall is located outside a beam emission end of the shielding chamber. A geometric center point of the accommodation cavity is set as a Bragg peak of the beam.

In some embodiments, the shielding chamber is a layered structure spirally assembled from the inside out in sequence. The shielding chamber includes a proton shielding layer and a neutron shielding layer. The proton shielding layer includes a plurality of proton shielding units spirally stacked from the inside out along a first preset spiral trajectory with the central axis of the beam as a center line. The neutron shielding layer includes a plurality of neutron shielding units spirally stacked around the center axis of the beam on an outside of the proton shielding layer along a second preset spiral trajectory.

In some embodiments, the plurality of proton shielding units and the plurality of neutron shielding units are spirally extended and spliced around the central axis of the beam to form a cylindrical structure. The first window and the second window are respectively located at both ends of the cylindrical structure. The both ends of the cylindrical structure are along the direction of the beam.

In some embodiments, a positioning mark is provided at the center point of the first window. The positioning mark is used to indicate the center point of the first window. The first window and the second window are located on opposite sides of the shielding chamber. The opposite sides of the shielding chamber are along the direction of the beam.

In some embodiments, a count of the plurality of proton shielding units is equal to a count of the plurality of neutron shielding units.

In some embodiments, a material of the plurality of proton shielding units includes at least one of stainless steel, lead, aluminum, or concrete.

In some embodiments, a material of the plurality of neutron shielding units includes at least one of polyethylene, polypropylene, polystyrene, polyester, boron, lead, tungsten, iron, or barium.

One or more embodiments of the present disclosure provide a radiation protection shielding device. The radiation protection shielding device includes a shielding chamber for radiation protection and a movable device. The shielding chamber for radiation protection includes an accommodation cavity, and a first window and a second window arranged sequentially at intervals along a direction of a beam on the shielding chamber. The movable device is used for adjusting a position of the shielding chamber so that a center point of the first window is aligned with an isocenter of the beam.

In some embodiments, the movable device includes a worktable and a drive module. The worktable configured to support the shielding chamber and drive the shielding chamber to move up and down. The drive module configured to provide power for the worktable.

In some embodiments, the movable device includes a lifting mechanism and a planar moving component. The lifting mechanism configured to adjust a height of the shielding chamber. The planar moving component configured to change the position of the shielding chamber on the worktable. The lifting mechanism and the planar moving component are configured to operate in coordination to make a geometric center point of the accommodation cavity of the shielding chamber coincide with a Bragg peak of the beam.

In some embodiments, the lifting mechanism is a hydraulic scissor lift. The drive module includes a hydraulic cylinder and a hydraulic unit. The lifting mechanism further includes a limit switch and a hydraulic handle.

In some embodiments, the hydraulic cylinder is connected to the shielding chamber. The hydraulic unit is configured to provide power to the hydraulic cylinder. The limit switch is configured to control a final position of lifting. The hydraulic handle is configured to manually control a lifting movement.

In some embodiments, the planar moving component is disposed on an upper surface of the worktable. The planar moving mechanism is a ball screw module.

In some embodiments, the first window and the second window are located on opposite sides of the shielding chamber. The worktable is connected to the shielding chamber through the ball screw module so that the shielding chamber moves back and forth along the direction of the beam.

In some embodiments, the ball screw module includes a handwheel and a screw. A bottom of the shielding chamber is slidably disposed on a guide rail on the upper surface of the worktable. The upper surface of the worktable is provided with two first screw seats. The bottom of the shielding chamber is provided with a second screw seat. One end of the screw is connected to the handwheel. The other end of the screw passes through a screw hole on one of the two first screw seats and the second screw seat, and is connected to another of the two first screw seats.

In some embodiments, the radiation protection shielding device further includes a position adjustment device. The position adjustment device is disposed within the accommodation cavity. The position adjustment device is configured to adjust and position a target object.

One or more embodiments of the present disclosure provide a FLASH therapy system. The FLASH therapy system includes a proton accelerator and a radiation protection shielding device. The proton accelerator is configured to output a beam. The radiation protection shielding device includes a shielding chamber for radiation protection and a movable device. The shielding chamber for radiation protection includes an accommodation cavity, and a first window and a second window arranged sequentially at intervals along a direction of a beam on the shielding chamber. The movable device is used for adjusting a position of the shielding chamber so that a center point of the first window is aligned with an isocenter of the beam.

In some embodiments, the FLASH therapy system further includes a first ionization chamber, a carbide absorber, a collimator, and a second ionization chamber arranged sequentially at intervals along the direction of the beam. The carbide absorber is configured to adjust an energy range extracted by the proton accelerator according to tumor information of a target object. The collimator is configured to calibrate the beam. The first ionization chamber is configured to measure a real-time dose of the beam and feed the real-time dose back to a dose control system. The second ionization chamber is configured to measure an absolute dose of the beam and is located in front of the first window.

In some embodiments, the target object includes an animal or human suffering from a tumor.

In some embodiments, the FLASH therapy system further includes a Faraday cup close to the second window, the Faraday cup being configured to measure a magnitude of the beam and collect a surplus beam.

Additional features will be partly explained in the description below, and will become apparent to those skilled in the art by reviewing the following content and drawings, or through the understanding of production or operation examples. The features of the embodiments described in this specification can be achieved and obtained by practicing or using various aspects of the methods, tools, and combinations detailed in the examples below.

### BRIEF DESCRIPTION OF THE DRAWINGS

The present disclosure will be further described by way of exemplary embodiments. These exemplary embodiments will be described in detail with reference to the accompanying drawings. These embodiments are not limiting. In these embodiments, the same reference numerals refer to the same structures, where:

FIG. 1 is a schematic diagram illustrating an exemplary shielding chamber for radiation protection according to some embodiments of the present disclosure;

FIG. 2 is a schematic diagram illustrating an exemplary shielding chamber for radiation protection according to other embodiments of the present disclosure;

FIG. 3 is a schematic diagram illustrating an exemplary radiation protection shielding device according to some embodiments of the present disclosure;

FIG. 4 is a schematic diagram illustrating an exemplary movable device according to some embodiments of the present disclosure;

FIG. 5 is a schematic diagram illustrating an exemplary movable device according to some other embodiments of the present disclosure;

FIG. 6 is a schematic diagram illustrating an exemplary position adjustment device according to some embodiments of the present disclosure;

FIG. 7 is a block diagram illustrating an exemplary FLASH therapy system according to some embodiments of the present disclosure;

FIG. 8 is a schematic diagram illustrating two types of exemplary machine rooms according to some embodiments of the present disclosure;

FIG. 9 is a schematic diagram illustrating radiation shielding simulation results of an exemplary machine room 1 according to some embodiments of the present disclosure;

FIG. 10 is a schematic diagram illustrating radiation shielding simulation results of an exemplary machine room 2 according to some embodiments of the present disclosure; and

FIG. 11 is a schematic flowchart illustrating an exemplary radiation protection shielding process according to some embodiments of the present disclosure.

### DETAILED DESCRIPTION

To more clearly illustrate the technical solutions in the embodiments of the present disclosure, the accompanying drawings required for describing the embodiments are briefly described below. Obviously, the accompanying drawings in the following description are merely some examples or embodiments of the present disclosure. For a person of ordinary skill in the art, the present disclosure may be applied to other similar scenarios based on these accompanying drawings without making creative efforts. Unless obvious from the context or otherwise indicated by the context, the same reference numerals in the drawings refer to the same structures or operations.

It should be understood that the terms “system”, “device”, “unit”, and/or “module” used herein are a method for distinguishing components, elements, parts, portions, or assemblies of different levels. However, if other terms can achieve the same purpose, the aforementioned terms may be replaced by other expressions.

As used in the present disclosure and the claims, unless the context clearly indicates an exception, the terms “a”, “an”, “one”, and/or “the” are not limited to the singular form and may include the plural form. Generally, the terms “include” and “comprise” merely indicate that the identified steps and elements are included. These steps and elements do not constitute an exclusive list, and a method or device may also include other steps or elements.

Flowcharts are used in the present disclosure to illustrate operations performed by systems according to the embodiments of the present disclosure. It should be understood that the preceding or following operations are not necessarily performed in exact sequence. On the contrary, the steps may be processed in reverse order or simultaneously. Meanwhile, other operations may be added to these processes, or one or more steps may be removed from these processes.

Proton tumor therapy technology is a relatively advanced, mature and high-end medical technology internationally at present, and is one of the main means for tumor treatment. Compared with high-energy neutrons and electrons widely used at present, protons may concentrate the energy of rays more effectively on the tumor target region requiring treatment, and improve the local tumor control rate. Meanwhile, protons may greatly reduce the probability of radiation complications occurring in normal organs and tissues. Proton therapy devices have become the mainstream equipment for tumor radiotherapy internationally at present.

FLASH radiotherapy mainly refers to the delivery of radiation dose to the entire treatment region within an ultra-short time (e.g., less than 1 second). Early preclinical evidence has shown that, compared with conventional radiotherapy, the extremely high dose rate of FLASH radiotherapy may significantly protect healthy tissues without reducing the damage to cancer cells. Since the FLASH high beam current delivers an enormous radiation dose in a very short time, extremely high requirements are put forward for radiation shielding walls. In other words, the site for conducting FLASH radiotherapy must have a sufficiently strong radiation shielding capacity. This requires the radiation shielding walls to be fabricated with sufficient thickness, which results in a large area occupied by the walls and entails a substantial capital investment.

Merely by way of example, in a commissioning room of a proton therapy system, the control level of dose rate outside a shielding body is shown in Table 1 according to radiation protection standards.

TABLE 1

| | | Position description | Dose rate control |
|---|---|---|---|
| Place | Position | (occupancy factor) | level, μSv/h |
| A | Outside | Occupancy factor | ≤2.5 |
| commissioning | surrounding | T > 1/2 | |
| room of a | shielding walls | Occupancy factor | ≤10 |
| proton therapy | of the | T ≤ 1/2 | |
| system | commissioning | | |
| | room, outside | | |
| | an entrance | | |
| | protective door, | | |
| | and on a top | | |
| | of the | | |
| | commissioning | | |
| | room | | |
| | Outside a | A junction between | ≤5 × 10³ |
| | bottom plate | an outer surface of | |
| | of the | the bottom plate of | |
| | commissioning | the commissioning | |
| | room | room and soil | |

The occupancy factor refers to a proportion of average residence time of personnel subject to maximum irradiation in a region to beam emission time during beam emission time of a radiation source. The dose rate refers to radiation energy absorbed per unit time, and the dose rate is usually measured in units of millisieverts or microsieverts. The dose rate is an important safety indicator configured to measure an impact of radiation on human health.

If a FLASH radiation protection shielding device is not provided, a very thick radiation shielding wall is required, which occupies a larger area of land and thus inevitably incurs huge capital costs ranging from millions to tens of millions of yuan.

Based on the above, embodiments of the present disclosure provide a shielding chamber for radiation protection, a radiation protection shielding device, and a FLASH therapy system. By designing the radiation protection shielding device, namely the FLASH radiation protection shielding device, the problem of radiation protection and shielding of a commissioning room during high beam current commissioning and experiments is solved. Meanwhile, the radiation protection shielding device satisfies the function of up-down and forward-backward adjustment during FLASH experiments, and allows a test target to be adjusted to an isocenter position of the device. On the premise of satisfying requirements for isocenter position adjustment and high beam current radiation shielding, the economic cost of a huge shielding wall required for conducting high beam current FLASH experiments is saved.

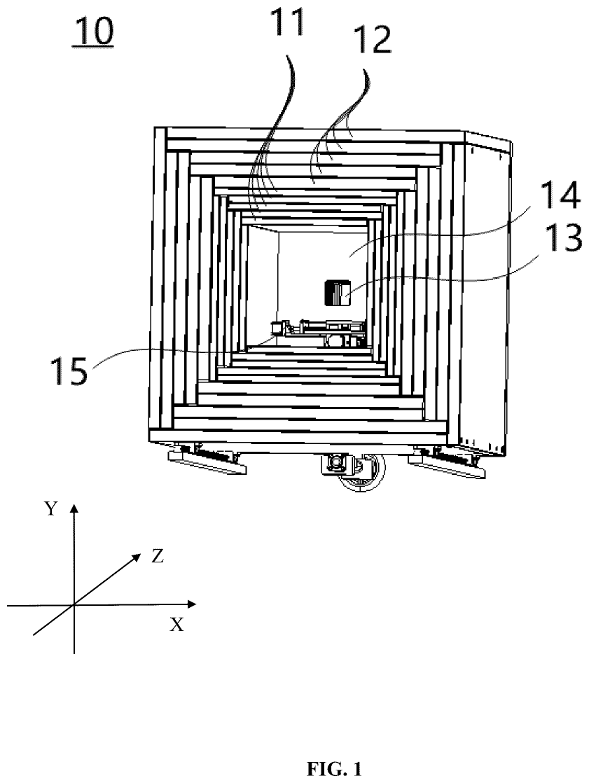

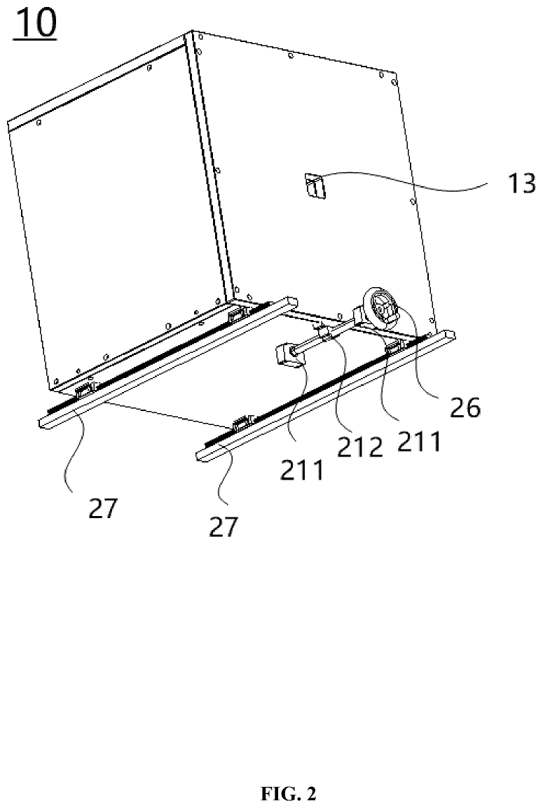

FIG. 1 is a schematic diagram illustrating an exemplary shielding chamber for radiation protection according to some embodiments of the present disclosure. FIG. 2 is a schematic diagram illustrating an exemplary shielding chamber for radiation protection according to other embodiments of the present disclosure.

As shown in FIG. 1 and FIG. 2, a shielding chamber 10 for radiation protection includes an accommodation cavity, and a first window 13 and a second window 14 arranged sequentially at intervals along a direction of a beam on the shielding chamber.

The shielding chamber is a protective device configured to shield radiation generated during radiotherapy. In some embodiments, the shielding chamber may absorb direct radiation of proton beams and attenuate secondary neutron radiation.

Embodiments of the present disclosure do not limit the dimensions of the shielding chamber 10. The length of the shielding chamber 10 along the beam emission direction may be 1000 mm, 1400 mm, 1800 mm or 2000 mm. The width of the shielding chamber 10 may be 800 mm, 1400 mm or 1600 mm. The adjustable lifting height range of the shielding chamber 10 may be 500 mm˜1500 mm. The load capacity of the shielding chamber 10 may be 3.2 T, 4.3 T, 4.6 T or 5 T. The width of the shielding chamber 10 refers to the maximum external dimension of the shielding chamber measured along the horizontal transverse direction perpendicular to the beam transmission direction. For example, the width of the shielding chamber 10 refers to the width along the X-axis direction as shown in FIG. 1. The adjustable lifting height range of the shielding chamber 10 refers to the displacement range of the shielding chamber 10 from the lowest working position to the highest working position. For example, the adjustable lifting height range of the shielding chamber 10 refers to the height along the Z-axis direction as shown in FIG. 1.

In some embodiments, as shown in FIG. 1 and FIG. 2, the shielding chamber 10 is a laminated structure assembled sequentially in a spiral manner from the inside out. In some embodiments, the shielding chamber 10 includes a proton shielding layer 11 and a neutron shielding layer 12 arranged sequentially from the inside out.

The proton shielding layer 11 is a structure configured to block and absorb proton beams output by a proton accelerator during radiotherapy (e.g., FLASH radiotherapy). In some embodiments, the proton shielding layer 11 is disposed on the inner layer of the shielding chamber 10. A beam refers to a stream of charged particles output by an accelerator. For example, a beam includes a proton beam output by a proton accelerator. A beam central axis refers to a reference straight line corresponding to the geometric center of a beam's motion trajectory.

A proton shielding unit refers to a component constituting the proton shielding layer 11. A proton shielding unit is a basic protective unit that forms a proton shielding layer. Proton shielding units form a continuous proton blocking barrier through laminated combination, and are capable of absorbing or blocking high-energy proton beams. In some embodiments, a single proton shielding unit may be independently disassembled and/or replaced. In some embodiments, the number of proton shielding units constituting the proton shielding layer 11 may be adjusted according to the beam dose.

In some embodiments, the proton shielding unit may be a proton shielding plate. In some embodiments, as shown in FIG. 1, the proton shielding layer 11 is formed by laminating a plurality of proton shielding units spirally from the inside out along a first preset spiral trajectory, with the beam central axis as the central line. The first preset spiral may be determined according to the dimensions of the proton shielding units. For example, the pitch of the preset spiral may be the thickness of a single proton shielding unit, the spiral direction may be clockwise (or counterclockwise), and the spiral lead angle may be determined according to the average diameter of the proton shielding units. For more descriptions about the proton shielding layer including a plurality of proton shielding units, reference may be made to the relevant sections hereinafter.

In some embodiments, a material of the proton shielding unit includes at least one of stainless steel, lead, aluminum, or concrete.

The neutron shielding layer 12 is a structure configured to block and absorb secondary neutron radiation generated by the interaction between proton beams and the proton shielding layer. In some embodiments, the neutron shielding layer 12 is disposed on the outer layer of the shielding chamber 10.

A neutron shielding unit refers to a component constituting the neutron shielding layer 12. A neutron shielding unit is a basic protective unit that forms a neutron shielding layer. Neutron shielding units form a continuous neutron blocking barrier through laminated combination, and are capable of attenuating secondary neutron radiation. In some embodiments, a single neutron shielding unit may be independently disassembled and/or replaced. In some embodiments, a count of the neutron shielding units included in the neutron shielding layer 12 may be adjusted according to the beam dose. In some embodiments, a neutron shielding unit may be a neutron shielding plate. In some embodiments, as shown in FIG. 1, the neutron shielding layer 12 is formed by laminating a plurality of neutron shielding units spirally on the outer side of the proton shielding layer along a second preset spiral trajectory, with the beam central axis as the central line. The second preset spiral may be determined according to the dimensions of the neutron shielding units. For example, the pitch of the preset spiral may be the thickness of a single neutron shielding unit, the spiral direction may be clockwise (or counterclockwise), and the spiral lead angle may be determined according to the average diameter of the neutron shielding units. For more descriptions about the neutron shielding layer including a plurality of neutron shielding units, reference may be made to the relevant sections hereinafter.

In some embodiments, a material of the plurality of neutron shielding units includes at least one of polyethylene, polypropylene, polystyrene, polyester, boron, lead, tungsten, iron, or barium.

In some embodiments of the present disclosure, proton shielding units are made of materials such as stainless steel, lead, aluminum, and concrete, while neutron shielding units are made of materials such as polyethylene, polypropylene, polystyrene, polyester, boron, lead, tungsten, iron, and barium. These materials are low in cost and easily accessible, which reduces the manufacturing cost and maintenance cost of the radiation protection shielding device on the premise of meeting radiation shielding requirements.

In some embodiments, the proton shielding layer 11 includes M proton shielding units laminated from the inside out. In some embodiments, the neutron shielding layer 12 includes N neutron shielding units laminated from the inside out, where both M and N are positive integers. In some embodiments, the proton shielding layer 11 and the neutron shielding layer 12 each include multiple layers. In some embodiments, a count of the layers of the proton shielding units in the proton shielding layer 11 and a count of the layers of the neutron shielding units in the neutron shielding layer 12 may be the same. For example, a count of the layers of both the proton shielding units and the neutron shielding units is 5. In some embodiments, for ease of description, the proton shielding units and the neutron shielding units may also be collectively referred to as shielding units.

In some embodiments of the present disclosure, providing the shielding chamber 10 with a laminated structure reduces the overall volume and weight of the shielding chamber 10 on the premise of meeting radiation shielding requirements. On the one hand, it facilitates the disassembly of proton shielding units and neutron shielding units; on the other hand, it facilitates the adjustment of the position of the shielding chamber 10 by the mobile device 20.

In some embodiments of the present disclosure, the proton shielding layer 11 and the neutron shielding layer 12 are formed by laminating the plurality of shielding units, which makes it possible to increase the number of shielding unit layers without increasing the overall thickness of the shielding chamber 10. Each layer of shielding units effectively absorbs and reduces radiation penetration, thereby further lowering the radiation level of the external environment. Each layer of shielding units is independently detachable, so the count of shielding units may be flexibly selected, increased or decreased according to specific conditions to meet the radiation shielding requirements in different application scenarios. The detachable proton shielding units and/or neutron shielding units enable the radiation protection shielding device to adapt to different therapeutic doses and radiation sources.

In some embodiments, the cross-sectional shape of the shielding chamber 10 perpendicular to the beam emission direction may be rectangular, annular, or other suitable shapes. In some embodiments, the proton shielding unit may be an arc-shaped plate or a rectangular plate. In some embodiments, the shape of the neutron shielding unit is the same as that of the proton shielding unit to ensure that the proton shielding layer and the neutron shielding layer fit closely together. For example, if the proton shielding unit is an arc-shaped plate, the neutron shielding unit is also an arc-shaped plate, and the cross-sectional shape of the shielding chamber 10 perpendicular to the beam emission direction is annular. For another example, if the proton shielding unit is a rectangular plate, the neutron shielding unit is also a rectangular plate, and the cross-sectional shape of the shielding chamber 10 perpendicular to the beam emission direction is rectangular.

In some embodiments, the proton shielding units and the neutron shielding units are spirally extended and spliced around the beam central axis to form a cylindrical structure. Herein, spiral extension and splicing refers to an installation method similar to a “spiral staircase” structure, where after the shielding units of the previous layer are spliced into a complete cylindrical structure, the units of the next layer will be offset by a fixed distance along the beam direction, and their circumferential starting positions will be staggered by a preset angle at the same time. The fixed offset distance may be determined according to the thickness of the shielding units. For example, the fixed offset distance may be equal to the thickness of the shielding units. The preset angles for staggering the circumferential starting positions include 5°, 10°, or 15°, etc. In some embodiments of the present disclosure, the spiral extension and splicing of the proton shielding units and the neutron shielding units around the beam central axis makes all the splicing gaps of the proton shielding layer and the neutron shielding layer distribute in a spiral and staggered manner, which avoids the coincidence of gaps between different shielding layers to form through gaps and improves the radiation shielding effect.

In some embodiments, screw holes or buckles are provided on the sides of the shielding units (e.g., proton shielding units and/or neutron shielding units). During installation, a plurality of proton shielding units are spirally laminated from the inside out along a first preset spiral trajectory with the beam central axis as the central line, and each proton shielding unit is fixed by connectors (e.g., bolts or special connecting rods) or buckles to form a cylindrical proton shielding layer. A plurality of neutron shielding units are spirally laminated on the outer side of the proton shielding layer along a second preset spiral trajectory with the beam central axis as the central line, and each neutron shielding unit is fixed by connectors (e.g., bolts or special connecting rods) or buckles to form a cylindrical neutron shielding layer.

In some embodiments, each shielding layer (e.g., the proton shielding layer and the neutron shielding layer) may also be pre-assembled into a cylindrical structure by the plurality of shielding units, which is then transported into the shielding chamber 10 for spiral extension and splicing around the beam central axis. For example, after the inner-layer shielding units are assembled into a complete cylindrical structure, the next-layer shielding units are installed with a fixed offset distance along the beam direction and a preset staggered angle at the circumferential starting position.

In some embodiments, multiple shielding units may also be connected by magnetic attraction for easy disassembly.

In some embodiments, the proton shielding layer 11 is formed by stacking five layers of stainless steel plates.

In some embodiments, the neutron shielding layer 12 is formed by stacking five layers of polyethylene (PE) plates.

In some embodiments, the shielding chamber 10 is composed of stainless steel plates and PE plates, where the stainless steel layers are configured to shield the protons generated by radiation scattering, and the PE layers are configured to shield the neutrons generated by radiation scattering.

The accommodation cavity is a closed/semi-closed space inside the shielding chamber 10. In some embodiments, the accommodation cavity may be used to carry a target object. Herein, the target object refers to the subject undergoing radiotherapy (e.g., FLASH radiotherapy). In some embodiments, the target objects include tumor-bearing animals or humans, etc. In some embodiments, the accommodation cavity is the hollow part of the cylindrical structure formed by the proton shielding units and the neutron shielding units. In some embodiments, the accommodation cavity is located between the first window and the second window, and the first window and the second window are respectively connected to the accommodation cavity.

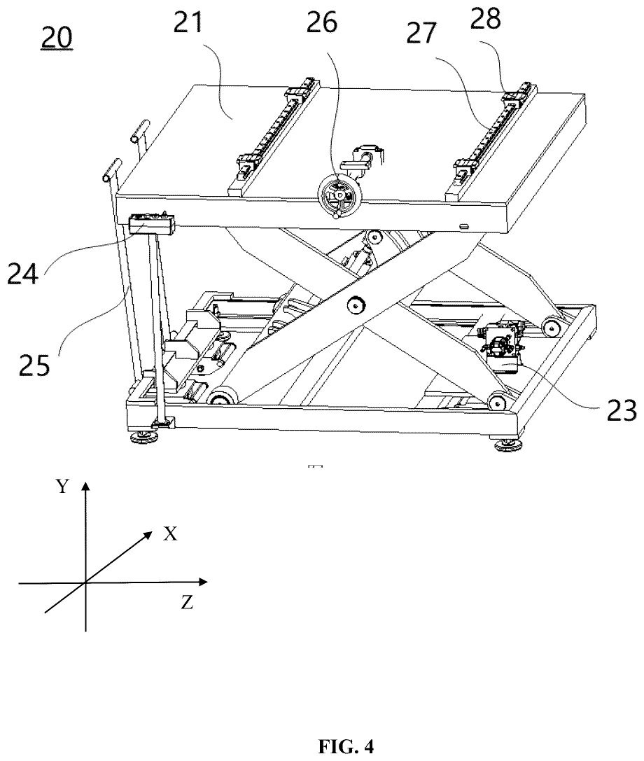

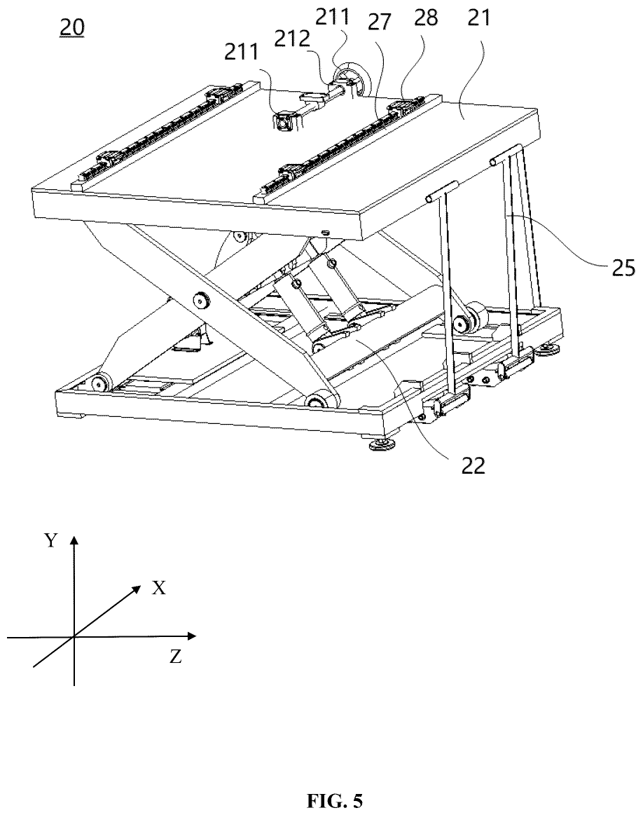

In some embodiments, the geometric center point of the accommodation cavity is preset as the Bragg peak position of the beam. The Bragg peak position refers to the position where a charged particle beam (e.g., proton beam) releases energy concentratedly at the end of its range to form a sharp dose peak after entering a medium (e.g., human tissue, experimental animal body, water-equivalent material). That is to say, the Bragg peak position is the spatial point where the proton beam releases the maximum energy in the target medium, and it is also the ideal irradiation center for the tumor target area in FLASH radiotherapy. In some embodiments, the shielding chamber is movable. In some embodiments, the geometric center point of the accommodation cavity may be set as the Bragg peak position of the beam by moving the shielding chamber, that is, the geometric center point of the accommodation cavity is aligned with the Bragg peak position of the beam through the movement of the shielding chamber. For more descriptions about setting the geometric center point of the accommodation cavity as the Bragg peak position of the beam, reference may be found in FIG. 4, FIG. 5, etc.

In some embodiments, no plates are spliced on the plane of the shielding chamber 10 facing away from the beam emission direction, thus naturally forming a relatively large window (i.e., the second window 14). A shielding plate is arranged on the plane of the shielding chamber 10 facing the beam incident direction, and a relatively small window is opened on the shielding plate, namely the first window 13. The shielding plate may include proton shielding units and neutron shielding units.

In some embodiments, the first window 13 and the second window 14 are respectively located at the two ends of the cylindrical structure of the shielding chamber 10 along the beam direction.

The first window 13 is an opening structure arranged on a side of the shielding chamber 10 adjacent to the proton accelerator (i.e., a beam incident end). Specifically, the first window 13 is located at the beam incident end of the shielding chamber 10. The beam incident end refers to the end of the shielding chamber 10 where the beam enters the shielding chamber 10.

The second window 14 is an opening structure disposed on the side of the shielding chamber 10 away from the proton accelerator (i.e., the beam exit end and/or the end for the target object to enter and exit the shielding chamber). That is to say, the second window 14 is located at the beam exit end of the shielding chamber 10. The beam exit end refers to the end from which the beam exits the shielding chamber 10.

In some embodiments, the center point of the first window 13, the center point of the second window 14, and the beam central axis lie on the same straight line.

In some embodiments, the first window 13 and the second window 14 are respectively in communication with the accommodation cavity.

In some embodiments, the first window 13 is used for entry of the beam. The size of the first window 13 is smaller than that of the second window 14.

In some embodiments, the second window is used for the target object to pass through, and the target object is accommodated in the accommodation cavity after passing through the second window.

The embodiment of the present disclosure does not limit the sizes and shapes of the first window 13 and the second window 14. The shapes of the first window 13 and the second window 14 may be the same or different. For example, the shapes of the first window 13 and the second window 14 may be rectangular, circular, regular polygonal, triangular, fan-shaped, or irregular.

The first window 13 needs to be aligned with the beam outlet, mainly to allow the beam to enter through the first window 13 and sufficiently contain the scattering of the beam during its flight. The second window 14 needs to be aligned with the wall. The size of the second window 14 may be flexibly set according to the size of the target object.

The first window 13 and the second window 14 can both be configured as squares. The side length of the first window 13 can be 50 mm, 80 mm, 100 mm, or 150 mm. The side length of the second window 14 can be 300 mm, 500 mm, 700 mm, or 800 mm.

In some embodiments, the shielding chamber 10 is disposed in the treatment room for radiation therapy. In some embodiments, a radiation shielding wall is arranged in the treatment room to further absorb and block the beam emitted from the shielding chamber 10. For example, the radiation shielding wall can absorb and block the beam emitted from the second window 14 of the shielding chamber 10. Herein, the radiation shielding wall is located outside the beam exit end of the shielding chamber. In some embodiments, the second window 14 is close to the radiation shielding wall of the treatment room. For example, the second window faces the radiation shielding wall of the treatment room, and the distance between the second window and the radiation shielding wall is less than a preset value. The distance between the second window and the radiation shielding wall can be determined according to the beam energy, dose rate, etc. For example, the higher the beam energy and the higher the dose rate, the closer the distance between the second window and the radiation shielding wall. In some embodiments of the present disclosure, the second window 14 is arranged in close contact with the radiation shielding wall of the treatment room, which can block the leakage path of the beam and secondary radiation, and improve the radiation protection effect of the shielding chamber 10. In some embodiments, the shielding chamber 10 is movable. Before radiation therapy is performed, the second window 14 is away from the radiation shielding wall of the treatment room. After the target object enters the accommodation cavity through the second window 14, the shielding chamber 10 is moved to make the second window 14 fit closely with the radiation shielding wall of the treatment room. After radiation therapy is completed, the shielding chamber 10 is moved to make the second window 14 away from the radiation shielding wall of the treatment room, so that the target object can leave the accommodation cavity through the second window 14.

In some embodiments, a positioning mark is provided at the center point of the first window. The positioning mark is used to indicate the center point of the first window. In some embodiments, the first window and the second window are located on opposite sides of the shielding chamber. For example, the first window 13 and the second window 14 are respectively located at the two ends of the cylindrical structure formed by the proton shielding units and the neutron shielding units along the beam direction.

In some embodiments, the positioning mark can be a crosshair. The ISO (isocenter) position of the equipment is located by means of an external laser lamp crosshair. A crosshair is engraved on the first window 13 (the polyethylene front panel). The external laser lamp crosshair is aligned with the engraved crosshair by adjusting the hydraulic handle 25 up and down, with an accuracy of ±1 mm, which meets the ISO positioning requirements.

In some embodiments of the present disclosure, the provision of a positioning mark enables the operator to accurately determine the position of the center point of the first window 13. Ensuring that the center point of the first window 13 is aligned with the isocenter of the beam helps to ensure that the beam is accurately delivered to the target object, improving the precision of the experiment or treatment.

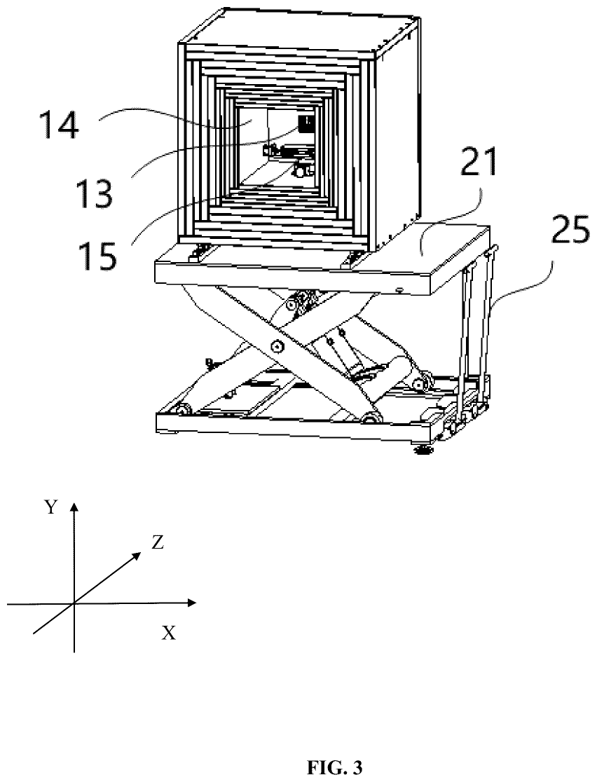

FIG. 3 is a schematic diagram illustrating an exemplary radiation protection shielding device according to some embodiments of the present disclosure. As shown in FIG. 3, the radiation protection shielding device 30 includes a shielding chamber 10 and a movable device 20.

The shielding chamber 10 is configured for radiation protection. In some embodiments of the present disclosure, the shielding chamber 10 includes an accommodation cavity and the first window 13 and the second window 14 arranged sequentially at intervals along a direction of a beam on the shielding chamber. More descriptions regarding the shielding chamber may be found in other contents of the present disclosure (e.g., descriptions in connection with FIGS. 1-2).

The movable device 20 is configured to adjust a position of the shielding chamber 10 so that a center point of the first window 13 is aligned with an isocenter of a beam. Herein, a center point of the first window 13 being aligned with an isocenter of a beam refers to that a distance between three-dimensional coordinates of the center point of the first window 13 and three-dimensional coordinates of the isocenter of the beam is less than a preset value.

A center point of a first window refers to a geometric center of the first window. Merely by way of example, the center point may be an intersection point of diagonals of a square window, a center of a circular window, or the like. The center point of the first window is a reference point for incidence of a beam.

An isocenter of a beam refers to an intersection point of a rotation axis of a treatment head and a central axis of the beam. Herein, the treatment head is a modular component integrated at a beam transmission end of a proton accelerator. The treatment head is configured to shape, calibrate, regulate a dose of, and modulate a range of a proton beam output by the proton accelerator, ensuring that parameters of the beam meet clinical application requirements for FLASH radiotherapy.

In some embodiments of the present disclosure, alignment of a center of the first window 13 of the shielding chamber 10 with an isocenter of a beam by using the movable device 20 ensures that the beam is accurately delivered to a tumor region of a target object, improving precision of FLASH radiotherapy. After completion of high-flux FLASH radiotherapy, the shielding chamber 10 may be moved to another storage position by using the movable device 20, which avoids a problem that a radiation shielding wall of a proton accelerator room needs to be added for implementation of high-flux FLASH radiotherapy. Under a premise of flexible operation, a huge amount of infrastructure economic cost and floor area cost are saved. In summary, the embodiments of the present disclosure not only meet radiation protection requirements for high-flux FLASH radiotherapy, but also fulfill a function of positioning the isocenter, reducing floor area and infrastructure costs.

FIG. 4 is a schematic diagram illustrating an exemplary movable device according to some embodiments of the present disclosure. FIG. 5 is a schematic diagram illustrating an exemplary movable device according to other embodiments of the present disclosure. In some embodiments, the movable device 20 includes a worktable 21 and a drive module.

A worktable 21 refers to a load-bearing structural component directly supporting a shielding chamber 10 in a movable device 20. In some embodiments, the worktable 21 is configured to support the shielding chamber 10 and drive the shielding chamber 10 to perform a lifting movement.

A drive module refers to an assembly of components configured to provide power for a lifting movement of a worktable 21.

In some embodiments of the present disclosure, flexible adjustment of a position of the shielding chamber 10 may be achieved through coordinated use of the worktable 21 and the drive module. Compared with a traditional fixed radiation shielding wall, use of the movable device 20 may save space, and a lifting movement of the worktable 21 may adjust the position of the shielding chamber 10 without requiring an excessive amount of floor area.

In some embodiments, the movable device 20 further includes a lifting mechanism and a planar moving component.

The lifting mechanism refers to an assembly of components for adjusting a vertical height of a shielding chamber in a movable device. In some embodiments, the lifting mechanism is configured to adjust a height of the shielding chamber.

In some embodiments, the lifting mechanism is a hydraulic scissor lift. The drive module includes a hydraulic cylinder 22 and a hydraulic unit 23. The lifting mechanism further includes a limit switch 24 and a hydraulic handle 25.

In one implementation manner, a weight of the movable device 20 may be 1.5 T, 1.8 T, or 2 T.

A model of the hydraulic cylinder 22 may be 63−35*150. The hydraulic unit 23 (also referred to as a hydraulic power unit or a hydraulic station) is connected to the hydraulic cylinder 22 through an external pipeline system to control actions of a plurality of groups of valves. A power of the hydraulic unit 23 may be 3 KW, a displacement of the hydraulic unit 23 may be 10 CC, and a pressure of the hydraulic unit 23 may be 12 Mpa.

In some embodiments, the hydraulic cylinder is connected to the shielding chamber. In some embodiments, the hydraulic unit is configured to provide power to the hydraulic cylinder. In some embodiments, the limit switch is configured to control a final position of lifting. In some embodiments, the hydraulic handle is configured to manually control a lifting movement.

In some embodiments, the hydraulic cylinder is connected to the shielding chamber in a hinged manner, which is configured to absorb slight vibrations during a lifting process, ensure that the shielding chamber lifts and lowers synchronously with the worktable without relative displacement, and guarantee positioning accuracy.

In some embodiments, the hydraulic unit is in communication with the hydraulic cylinder through an oil pipe, and outputs hydraulic oil of a corresponding pressure according to an operation instruction of the hydraulic handle to push a piston rod to extend and retract, achieving lifting and lowering of the shielding chamber.

In some embodiments of the present disclosure, the hydraulic scissor lift provides a stable lifting movement, which ensures that the shielding chamber 10 remains stable during an adjustment process and avoids unnecessary vibration and shaking. Specifically, the drive module includes the hydraulic cylinder 22 and the hydraulic unit 23, which is configured to precisely control a speed and a position of the lifting movement, making lifting adjustment of the shielding chamber 10 more accurate and controllable. By providing the limit switch 24 and the hydraulic handle 25, an operator may control the lifting movement through the limit switch 24 and the hydraulic handle 25, making the adjustment process more convenient and efficient.

In some embodiments, the movable device 20 further includes a planar moving component disposed on an upper surface of the worktable 21, and the planar moving component is configured to change a position of the shielding chamber 10 on the worktable 21.

In some embodiments, the planar moving component is a ball screw module. In some embodiments, the first window 13 and the second window 14 are located on opposite sides of the shielding chamber 10. In some embodiments, the worktable 21 is connected to the shielding chamber 10 through the ball screw module to enable the shielding chamber 10 to move back and forth along a movement direction of the beam.

By providing the ball screw module, back-and-forth movement of the shielding chamber 10 along a movement direction of the beam can be achieved. The ball screw module is capable of providing precise linear motion, ensuring more accurate position adjustment of the shielding chamber 10.

In some embodiments, the ball screw module is provided with a handwheel 26 and a lead screw.

A bottom portion of the shielding chamber 10 is slidably disposed on a guide rail 27 on an upper surface of the worktable 21. In some embodiments, two first lead screw seats 211 are disposed on the upper surface of the worktable 21. In some embodiments, a second lead screw seat 212 is disposed on the bottom portion of the shielding chamber 10. In some embodiments, one end of the lead screw is connected to the handwheel 26. In some embodiments, the other end of the lead screw passes through screw holes on one of the first lead screw seats 211 and the second lead screw seat 212 in sequence, and is connected to the other first lead screw seat 211.

Thus, the handwheel 26, serving as a control device, provides an intuitive operation mode, and an operator may rotate the handwheel 26 to control movement of the shielding chamber 10, featuring simple and clear operation. Specifically, the ball screw module is controlled through the handwheel 26, and a forward and backward movement distance of the shielding chamber 10 may be controlled according to a rotation angle. This precise control mode allows the operator to accurately adjust the position of the shielding chamber 10. In addition, the control mode of the handwheel 26 enables a quick response in the adjustment process, satisfying immediate requirements in treatment or tests.

In some embodiments, the movable device 20 is further provided with a slider 28 and a guide rail 27. The slider 28 is disposed on a lower surface of the shielding chamber 10, and the guide rail 27 is disposed on an upper surface of the worktable. The guide rail 27 is arranged in parallel with a traveling direction of the ball screw module. Clockwise and counterclockwise rotation of the handwheel 26 drives the shielding chamber 10 to perform a linear forward and backward movement.

In some embodiments, the lifting mechanism and the planar moving component operate in coordination to align a geometric center point of an accommodation cavity of the shielding chamber with a Bragg peak position of the beam.

In some embodiments, the radiation protection shielding device further includes a processing device (such as a central processing unit (CPU)). An operator may control the radiation protection shielding device through the processing device.

In some embodiments, a Bragg peak position of the beam is associated with a beam energy. In some embodiments, if the beam energy is a fixed value (such as 230 MeV), a fixed installation position of the shielding chamber may be preset to align a geometric center point of an accommodation cavity of the shielding chamber with the Bragg peak position of the beam. In some embodiments, the beam energy range is adjustable (for example, the beam energy range is 100 MeV-300 MeV) to adapt to tumor target areas at different depths. The geometric center point of the accommodation cavity of the shielding chamber may be adjusted by the movable device to align the geometric center point of the accommodation cavity of the shielding chamber with the Bragg peak position of the beam. For example, during a radiotherapy process, a beam energy value may be determined in advance, and after the beam energy value is determined, the position of the shielding chamber may be adjusted by the movable device to align the geometric center point of the accommodation cavity with the Bragg peak position of the beam. In some embodiments, after the geometric center point of the accommodation cavity is aligned with the Bragg peak position of the beam, the position of the shielding chamber may be fixed during the radiotherapy process.

In some embodiments, aligning the geometric center point of the accommodation cavity of the shielding chamber with the Bragg peak position of the beam includes the steps of: determining the Bragg peak position; and adjusting the geometric center of the accommodation cavity through the coordinated operation of the lifting mechanism and the planar moving component. Exemplarily, the Bragg peak position may be determined based on information such as beam parameters (e.g., beam energy, dose rate), the depth of the tumor target area of the target object (the depth along the beam direction, i.e., the distance from the tumor target area to the first window), the lateral position of the tumor center (in the X-axis direction), and the body position height during treatment (in the Y-axis direction). An operator may send an instruction to the hydraulic unit through the processing device to drive the hydraulic cylinder to drive the lifting and lowering of the worktable until the height of the geometric center point of the accommodation cavity reaches the Y-axis coordinate of the Bragg peak position. When the height of the geometric center point of the accommodation cavity reaches the Y-axis coordinate of the Bragg peak position, the limit switch immediately cuts off the hydraulic circuit to achieve mechanical locking, avoiding slight settlement during the lifting process. The operator may also drive the ball screw module through the processing device, and drive the shielding chamber to move back and forth along the Z-axis via the handwheel or a micro stepping motor until the Z-axis coordinate of the geometric center point of the accommodation cavity coincides with the Z-axis coordinate of the Bragg peak position. The operator may also send an instruction to the planar moving component through the processing device to drive the shielding chamber to move left and right along the X-axis until the X-axis coordinate of the geometric center point of the accommodation cavity coincides with the X-axis coordinate of the Bragg peak position, so as to align the geometric center point of the accommodation cavity of the shielding chamber with the Bragg peak position of the beam.

Traditionally, to meet radiation shielding requirements, FLASH radiotherapy requires the construction of heavy radiation shielding walls, resulting in high capital investment and increased floor space occupation. The embodiments of the present disclosure provide the movable device 20 and the shielding chamber 10, enabling the radiation protection shielding device to be adjusted in position according to treatment needs. In this way, there is no need to construct excessively heavy fixed shielding walls, which saves space and costs.

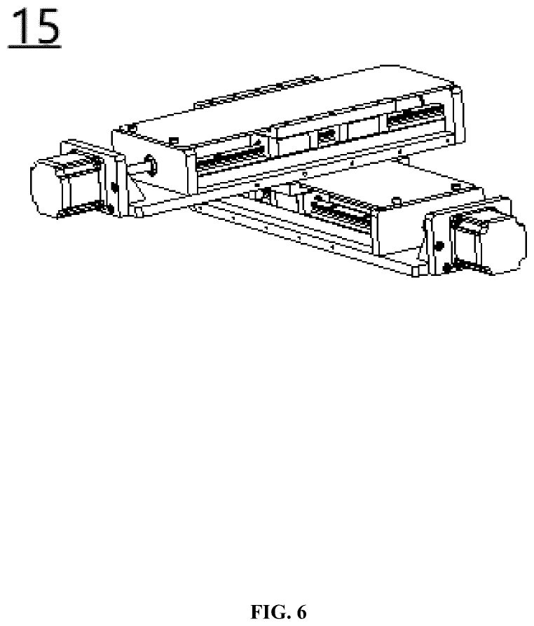

FIG. 6 is a schematic diagram illustrating an exemplary position adjustment device according to some embodiments of the present disclosure.

In some embodiments, a position adjustment device 15 is disposed in an accommodation cavity of the radiation protection shielding device, and the position adjustment device 15 is configured to adjust and position a target object.

In some embodiments, a position adjustment device 15 is configured to serve as a treatment couch for a target object, and the position adjustment device 15 is provided with a first guide rail capable of driving the target object to move back and forth, and a second guide rail capable of driving the target object to move left and right.

By providing a position adjustment device 15, a position of a target object may be adjusted and positioned, thereby ensuring that a beam can be accurately delivered to a tumor region of the target object.

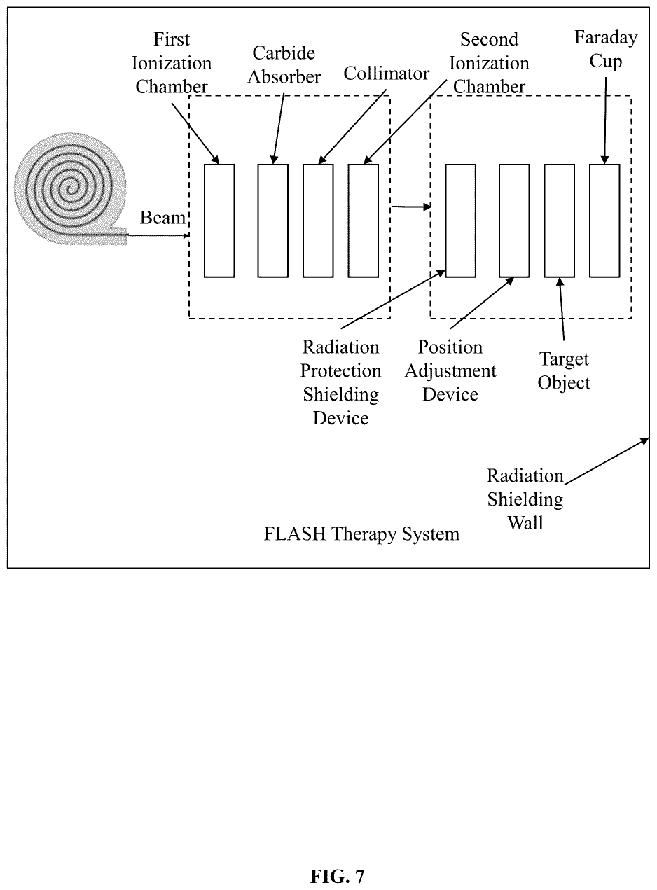

FIG. 7 is a schematic diagram illustrating a structural block diagram of an exemplary FLASH therapy system according to some embodiments of the present disclosure. The embodiments of the present disclosure also provide a FLASH therapy system. The FLASH therapy system includes a proton accelerator and a radiation protection shielding device. The proton accelerator is configured to output a beam.

Embodiments of the present disclosure do not impose limitations on a proton accelerator, and the proton accelerator may adopt an isochronous cyclotron or a synchrocyclotron.

A traditional cyclotron includes an isochronous cyclotron and a (pulsed) synchrocyclotron. In some embodiments, a beam of the isochronous cyclotron is quasi-continuous (a non-pulsed structure), while a beam of the synchrocyclotron is pulsed (a pulsed structure), which is an inherent and unique characteristic of a synchrocyclotron system. An instantaneous dose rate and an average dose rate of a pulsed output of the synchrocyclotron have unique advantages and effects for FLASH radiation in proton therapy.

In some embodiments, a cyclotron includes five subsystems, and the five subsystems include an ion source subsystem, a radio frequency (RF) subsystem, a vacuum subsystem, a magnet subsystem, and an extraction subsystem. Through complex interactions among the five subsystems, a proton beam for clinical treatment or research is generated. In terms of appearance, a cyclotron is usually shaped like a cylinder, and has two semicircular cylindrical electrode boxes with a small distance between them. The radio frequency (RF) subsystem forms an alternating electric field through the interval to accelerate protons when the protons pass through the interval. A dipole divides the cylinder into two independent parts, with one electrode shaped like the letter “D” and the other electrode shaped like an inverted letter “D”, which is the reason why the dipole electrodes are commonly known as “D-shaped electrodes”. A magnetic field is provided in the D-shaped electrodes to guide particles (i.e., protons) to travel from one D-shaped electrode to the other D-shaped electrode, and the protons also gain energy when passing through the slit.

A proton turns around and travels along a curved path back to the slit and the opposite D-shaped electrode. The proton passes through the slit and is accelerated, then enters the opposite D-shaped electrode, and travels along a curved path back to the slit and the opposite D-shaped electrode again. This process is repeated continuously until the proton reaches an extraction point near an outer edge of one of the D-shaped electrodes. At this time, an extraction subsystem guides a proton beam to enter a downstream subsystem, so that the proton beam can be further modified until it finally enters a target region inside a patient or a target object. Since the proton is accelerated when passing through the slit, the entire path traveled by the proton is an outwardly extending spiral path.

In some embodiments, the proton accelerator adopts a pulsed synchrocyclotron.

A pulsed synchrocyclotron can deliver protons at an ultra-high dose rate and accelerate the protons to 230 MeV (with a range of 32.0 g/cm², which is used for potential FLASH radiotherapy experiments, the time between pulses is 1.3 ms, and the duration of an ion source pulse is about 21 μs). Corresponding to a pulse with a repetition frequency of 750 Hz at a FLASH dose rate, an average dose rate measured at a Bragg peak is about 100 Gy/s, and an instantaneous dose rate is about 6200 Gy/s. The instantaneous dose rate is so high that although it has good advantages for FLASH therapy and research, it imposes high requirements on a radiation protection shielding device in a FLASH therapy system. Therefore, it is particularly crucial and necessary to design a radiation protection shielding method and related devices for a FLASH therapy system.

In some embodiments, the FLASH therapy system further includes a first ionization chamber, a carbide absorber, a collimator, and a second ionization chamber arranged sequentially at intervals along a direction of a beam.

In some embodiments, the carbide absorber is configured to adjust an energy range extracted by the proton accelerator according to tumor information of a target object. The target object is an animal or a human suffering from a tumor.

The collimator is configured to calibrate a beam.

The first ionization chamber is configured to measure a real-time dose of a beam, and feed the real-time dose back to a dose control system.

The second ionization chamber is configured to measure an absolute dose of a beam and is located in front of the first window.

In some embodiments, the FLASH therapy system further includes a Faraday cup close to the second window, and the Faraday cup is configured to measure a magnitude of a beam and collect a surplus beam.

The first ionization chamber is a real-time dose ionization chamber, which is configured to monitor and test a magnitude of a beam online, and feed back the magnitude to a dose control system rapidly. The carbide absorber is configured to adjust an energy range extracted by the proton accelerator according to tumor conditions of a target object. The collimator is configured to calibrate a surplus beam to make an edge of the beam sharper. The second ionization chamber is an absolute dose ionization chamber, which is configured to measure an absolute dose of the beam. The Faraday cup is configured to measure a magnitude of the beam and collect and block the surplus beam.

A plurality of studies on differential effects of high doses and low doses on proton radiotherapy have shown that a higher dose rate (usually exceeding 40 Gy/s) can affect normal tissues, and treatment at a rate with a total treatment time of less than 1 second exhibits less normal tissue toxicity while maintaining typical tumor control of traditional treatment. Industry studies have shown that FLASH is a revolutionary discovery in the field of radiotherapy. In application of proton therapy, it is important to consider combination of these studies with conformation provided by a Bragg peak. A conventional dose is generally in a range of 40 Gy to 100 Gy. To evaluate an impact of a FLASH dose rate within a spread-out Bragg peak (SOBP), a dosimetry method for measuring the FLASH dose rate and the conventional dose rate is designed.

Embodiments of the present disclosure adopt active and passive technologies of a dosimetry method to achieve redundant and effective beam control. A large number of literatures focus on the response characteristics of dosimeters to ultra-high dose rate delivery, including discussions on recombination effects in parallel-plate ionization chambers and film dose rate effects.

In some embodiments, the collimator adopts a plane-parallel ionization chamber (PPC05), which is configured to record a total dose and a dose rate at a delivery position before in-vivo use.

The PPC05 is configured to measure an absolute dose at an entrance depth and a Bragg peak. A bias voltage is set to 150 V, 300 V, 450 V, and +450 V in sequence. Generally speaking, a maximum bias voltage is 500 V. A surface of a diameter chamber is fabricated from a chamber holder model made of polycarbonate. Therefore, an effective measurement point is 2.3 mm WET. To perform measurements at a Bragg peak, an effective measurement point of the chamber holder model is 4.13 cm, and a sensitive volume for measurement in a Bragg peak ionization chamber is longitudinally located at the Bragg peak.

A first ionization chamber and/or a second ionization chamber adopts a tissue-equivalent ionization chamber (TIC).

The TIC and fixed film measurement are used as redundant measurements, and the TIC is generally connected to a dosimetry system, which is configured to measure and control a dose in real time.

Embodiments of the present disclosure adopt a synchrocyclotron for beam generation, and the beam is a single scattering spot with an energy of 230 MeV. To deliver a dose at the Bragg peak, a target object is placed in a boron carbide absorbing material with a water-equivalent thickness (WET) of approximately 30 cm, and a collimator with a diameter about twice the size of the beam spot. In addition, a small thickness of WET polyethylene absorbing material is used to adjust the beam delivery depth, allowing the target object and the absorbing material to be positioned at a configurable distance from the apparent source of radiation and centered horizontally. To treat a large clinically relevant target volume using a FLASH therapy system, it is necessary to expand the beam laterally and in depth while maintaining a local dose rate. In practical application, a ridge filter can be adopted, which is placed upstream of the boron carbide absorbing material to modify the depth profile of the beam to generate a spread-out Bragg peak (SOBP) without active modulation. Specifically, the ridge filter includes a grid of machined holes passing through an acrylic plastic (polymethyl methacrylate (PMMA)) block.

The initial average kinetic energy of protons is set to 230 MeV with an energy spread of 0.3%. The beam spot size at the beam extraction port ranges from 4 to 5 mm. The treatment isocenter in air and the beam energy spread are no higher than 1.5 mrad. A boron carbide block of a certain length is placed downstream of the cyclotron, followed by irradiation with a 1-cm-thick brass collimator. The square opening on the collimator is larger than the diameter of the brass collimator itself, and the collimator is configured to calibrate the beam that has undergone large-angle Coulomb scattering generated by the proton absorber.

In some embodiments, a target object can be a small animal (e.g., a mouse), which is anesthetized prior to beam irradiation and maintained under continuous anesthesia throughout the experimental operation. By viewing images collected in real time, it is ensured that the target object does not exhibit any obvious movement during treatment. If the target object is another type of subject, it is also necessary to ensure that it remains stationary during beam delivery. The target object can be a small animal, and it can also be a patient in need of proton radiotherapy. Among these components, a Faraday cup is a metal vacuum detector designed in a cup shape, which is used to measure the incident intensity of charged particles. The measured electric current can be used to determine the number of incident electrons or ions. For example, the target object can be an animal or a human suffering from a tumor.

A Faraday cup is configured to measure the flux or flux density in a proton beam. A Faraday cup includes a shielded insulating block with a thickness sufficient to block incident protons. The electric charge deposited in the Faraday cup is proportional to the number of protons stopped in the block, thus corresponding to the total flux of incident protons.

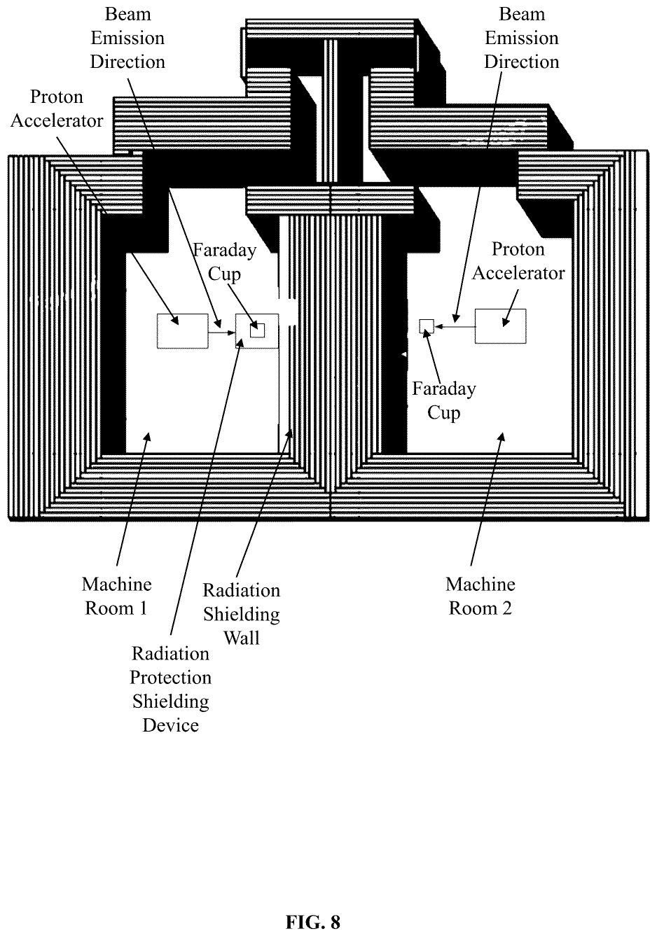

Referring to FIG. 8, FIG. 8 is a schematic diagram illustrating two types of exemplary machine rooms according to some embodiments of the present disclosure.

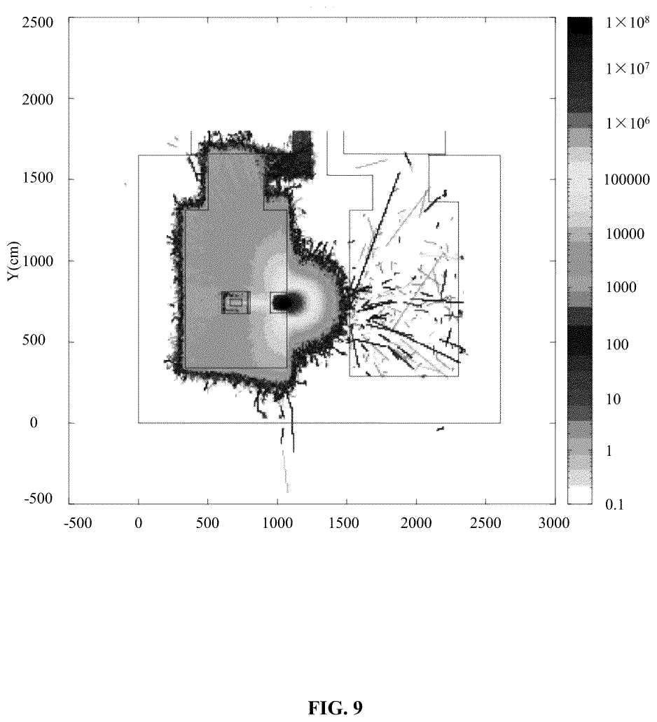

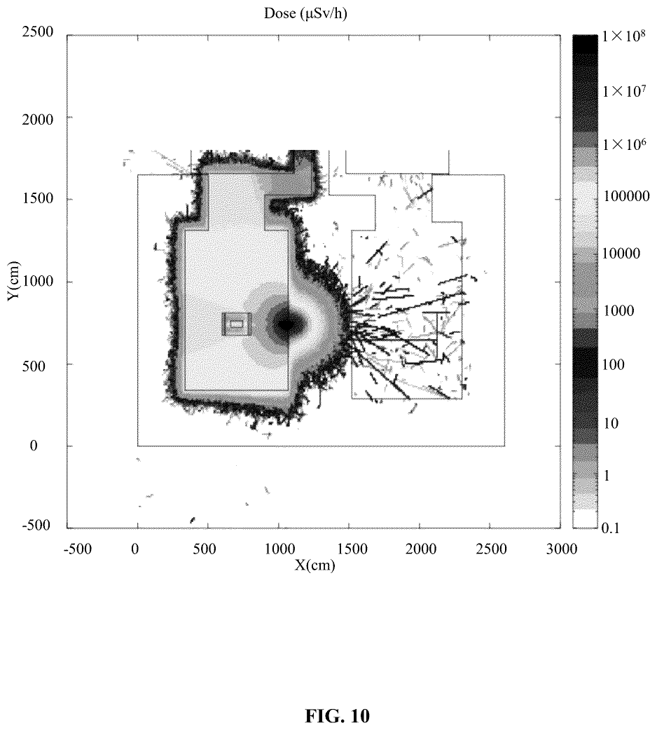

In a specific application scenario, a FLASH test is conducted in a proton commissioning machine room to verify a comparison of radiation shielding between a condition without the radiation protection shielding device and a condition with the radiation protection shielding device, and results are shown in FIGS. 9-10.

FIG. 9 is a schematic diagram illustrating a simulation result of radiation shielding for an exemplary machine room 1 according to some embodiments of the present disclosure.

FIG. 10 is a schematic diagram illustrating a simulation result of radiation shielding for an exemplary machine room 2 according to some embodiments of the present disclosure.

It may be seen that a dose difference inside a commissioning machine room between a condition with the radiation protection shielding device (machine room 1) and a condition without the radiation protection shielding device (machine room 2) is approximately three orders of magnitude. If a commissioning machine room is not provided with the radiation protection shielding device, a thickness of walls of the machine room needs to be about twice the actual thickness, so that an overall building size of the machine room is bound to increase greatly, and a more detailed design needs to be made for radiation monitoring in terms of radiation protection.

A detailed description of a radiation protection shielding method is provided below.

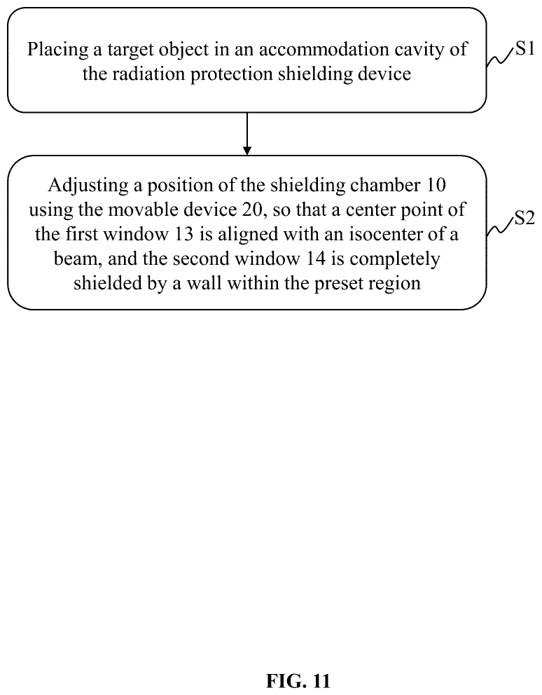

Referring to FIG. 11, FIG. 11 is a schematic diagram illustrating an exemplary process of a radiation protection shielding method according to some embodiments of the present disclosure.

Embodiments of the present disclosure further provide a radiation protection shielding method, which adopts any one of the aforementioned radiation protection shielding devices to perform radiation shielding within a preset region, and the method includes following steps.

In S1, a target object is placed in an accommodation cavity of the radiation protection shielding device.

In S2, a position of the shielding chamber 10 is adjusted by using the movable device 20, so that a center point of the first window 13 is aligned with an isocenter of a beam, and the second window 14 is completely shielded by a wall within the preset region.

Furthermore, a target object (e.g., a test animal or a Faraday cup) is placed in an accommodation cavity of the radiation protection shielding device, and a position of the shielding chamber 10 is adjusted so that a center point of the first window 13 of the radiation protection shielding device is accurately aligned with an isocenter of a beam, which can ensure that the beam is accurately delivered to the target object. In addition, the second window 14 is completely shielded, which can prevent radiation from leaking to an outside of the preset region and ensure safety of a treatment or a test process.

In some embodiments, the method further includes following steps.

Preset thicknesses of a proton shielding layer 11 and a neutron shielding layer 12 are obtained respectively according to a dose corresponding to a beam.

Preset thicknesses of the proton shielding layer 11 are used to set a count of the proton shielding units and a thickness of each proton shielding unit.

Preset thicknesses of the neutron shielding layer 12 are used to set a count of the neutron shielding units and a thickness of each neutron shielding unit.

Furthermore, preset thicknesses of shielding units of a proton shielding layer 11 and a neutron shielding layer 12 are accurately set according to a dose of a beam, which can optimize a shielding effect to the maximum extent. Meanwhile, a count and a thickness of the shielding units are accurately set, which can maximize utilization of resources, avoid excessive consumption of materials, and reduce manufacturing costs.

Embodiments of the present disclosure further provide a computer-readable storage medium, specific embodiments of the computer-readable storage medium are consistent with the embodiments described in the aforementioned method embodiments and technical effects achieved thereby, and redundant description of some contents is omitted.

The computer-readable storage medium stores a computer program, and the computer program, when executed by one or more processors, implements steps of any one of the aforementioned methods.

Embodiments of the present disclosure provide a program product, which is configured to implement steps of any one of the aforementioned methods. The program product may adopt a portable compact disc read-only memory (CD-ROM) and include program codes, and may run on a terminal device, such as a personal computer. However, the program product is not limited thereto. In one implementation, a readable storage medium may be any tangible medium that contains or stores a program, and the program may be used by or in combination with an instruction execution system, apparatus, or device. The program product may adopt any combination of one or more readable media. A readable medium may be a readable signal medium or a readable storage medium. A readable storage medium may be, for example but not limited to, an electronic, magnetic, optical, electromagnetic, infrared, or semiconductor system, apparatus, or device, or any combination of the above. More specific examples (a non-exhaustive list) of the readable storage medium include: an electrical connection having one or more wires, a portable disk, a hard disk, a random access memory (RAM), a read-only memory (ROM), an erasable programmable read-only memory (EPROM or flash memory), an optical fiber, a portable compact disc read-only memory (CD-ROM), an optical storage device, a magnetic storage device, or any suitable combination of the above.

A computer-readable storage medium may include a data signal that is contained in a baseband or propagated as part of a carrier wave, and in which readable program code is carried. Such a propagated data signal may adopt various forms, including but not limited to an electromagnetic signal, an optical signal, or any suitable combination of the above. The computer-readable storage medium may also be any readable medium that can send, propagate, or transmit a program for use by or in combination with an instruction execution system, apparatus, or device. Program code contained on the computer-readable storage medium may be transmitted using any appropriate medium, including but not limited to wireless, wired, optical fiber cable, RF, or any suitable combination of the above. Program code for performing operations of the present disclosure may be written in any combination of one or more programming languages, including object-oriented programming languages such as Java, C++, etc., and also conventional procedural programming languages such as the C language or similar programming languages. The program code may execute entirely on a user computing device, partially on a terminal device, as a stand-alone software package, partially on a user computing device and partially on a remote computing device, or entirely on a remote computing device or server. In cases involving a remote computing device, the remote computing device may be connected to the user computing device through any type of network, including a local area network (LAN) or a wide area network (WAN), or may be connected to an external computing device (e.g., connected through the Internet using an Internet service provider).

The basic concepts have been described above, and it is apparent to a person skilled in the art that the above detailed disclosure is intended only as an example and does not constitute a limitation of the present disclosure. While not expressly stated herein, various modifications, improvements, and amendments may be made to this disclosure by those skilled in the art. Those types of modifications, improvements, and amendments are suggested in this disclosure, so those types of modifications, improvements, and amendments remain within the spirit and scope of the exemplary embodiments of this disclosure.

What is claimed is:

1. A shielding chamber for radiation protection, comprising an accommodation cavity and a first window and a second window arranged sequentially at intervals along a direction of a beam on the shielding chamber; wherein:
the accommodation cavity is located between the first window and the second window, the first window and the second window are respectively in communication with the accommodation cavity, and a center point of the first window, a center point of the second window, and a central axis of the beam are on the same straight line;
the first window is used for entry of the beam, and a size of the first window is smaller than a size of the second window;
the second window is used for a target object to pass through, and the target object is accommodated in the accommodation cavity after passing through the second window; and
the shielding chamber is located inside a treatment room, the second window is close to a radiation shielding wall of the treatment room, the radiation shielding wall is located outside a beam emission end of the shielding chamber, and a geometric center point of the accommodation cavity is set as a Bragg peak of the beam.

2. The shielding chamber according to claim 1, wherein the shielding chamber is a layered structure spirally assembled from the inside out in sequence, the shielding chamber comprising:
a proton shielding layer including a plurality of proton shielding units spirally stacked from the inside out along a first preset spiral trajectory with the central axis of the beam as a center line; and
a neutron shielding layer including a plurality of neutron shielding units spirally stacked around the center axis of the beam on an outside of the proton shielding layer along a second preset spiral trajectory.

3. The shielding chamber according to claim 2, wherein the plurality of proton shielding units and the plurality of neutron shielding units are spirally extended and spliced around the central axis of the beam to form a cylindrical structure, the first window and the second window are respectively located at both ends of the cylindrical structure, and the both ends of the cylindrical structure are along the direction of the beam.

4. The shielding chamber according to claim 3, wherein a positioning mark is provided at the center point of the first window, the positioning mark is used to indicate the center point of the first window, the first window and the second window are located on opposite sides of the shielding chamber, and the opposite sides of the shielding chamber are along the direction of the beam.

5. The shielding chamber according to claim 4, wherein a count of the plurality of proton shielding units is equal to a count of the plurality of neutron shielding units.

6. The shielding chamber according to claim 5, wherein a material of the plurality of proton shielding units includes at least one of stainless steel, lead, aluminum, or concrete.

7. The shielding chamber according to claim 5, wherein a material of the plurality of neutron shielding units includes at least one of polyethylene, polypropylene, polystyrene, polyester, boron, lead, tungsten, iron, or barium.

8. A radiation protection shielding device, comprising:
a shielding chamber for radiation protection including an accommodation cavity, and a first window and a second window arranged sequentially at intervals along a direction of a beam on the shielding chamber; and
a movable device for adjusting a position of the shielding chamber so that a center point of the first window is aligned with an isocenter of the beam.

9. The radiation protection shielding device according to claim 8, wherein the movable device includes:
a worktable configured to support the shielding chamber and drive the shielding chamber to move up and down; and
a drive module configured to provide power for the worktable.

10. The radiation protection shielding device according to claim 9, wherein the movable device includes:
a lifting mechanism configured to adjust a height of the shielding chamber; and
a planar moving component configured to change the position of the shielding chamber on the worktable;
wherein the lifting mechanism and the planar moving component are configured to operate in coordination to make a geometric center point of the accommodation cavity of the shielding chamber coincide with a Bragg peak of the beam.

11. The radiation protection shielding device according to claim 10, wherein the lifting mechanism is a hydraulic scissor lift, the drive module includes a hydraulic cylinder and a hydraulic unit, and the lifting mechanism further includes a limit switch and a hydraulic handle.

12. The radiation protection shielding device according to claim 11, wherein the hydraulic cylinder is connected to the shielding chamber, the hydraulic unit is configured to provide power to the hydraulic cylinder, the limit switch is configured to control a final position of lifting, and the hydraulic handle is configured to manually control a lifting movement.

13. The radiation protection shielding device according to claim 10, wherein the planar moving component is disposed on an upper surface of the worktable; and the planar moving mechanism is a ball screw module.

14. The radiation protection shielding device according to claim 13, wherein the first window and the second window are located on opposite sides of the shielding chamber, and the worktable is connected to the shielding chamber through the ball screw module so that the shielding chamber moves back and forth along the direction of the beam.

15. The radiation protection shielding device according to claim 14, wherein the ball screw module includes a handwheel and a screw; a bottom of the shielding chamber is slidably disposed on a guide rail on the upper surface of the worktable; the upper surface of the worktable is provided with two first screw seats, and the bottom of the shielding chamber is provided with a second screw seat; one end of the screw is connected to the handwheel, and the other end of the screw passes through a screw hole on one of the two first screw seats and the second screw seat, and is connected to another of the two first screw seats.

16. The radiation protection shielding device according to claim 8, further comprising a position adjustment device; wherein the position adjustment device is disposed within the accommodation cavity, and the position adjustment device is configured to adjust and position a target object.

17. A FLASH therapy system, comprising: a proton accelerator and a radiation protection shielding device, wherein the proton accelerator is configured to output a beam; and the radiation protection shielding device includes:
a shielding chamber for radiation protection including an accommodation cavity, and a first window and a second window arranged sequentially at intervals along a direction of a beam on the shielding chamber; and
a movable device for adjusting a position of the shielding chamber so that a center point of the first window is aligned with an isocenter of the beam.

18. The FLASH therapy system according to claim 17, further comprising a first ionization chamber, a carbide absorber, a collimator, and a second ionization chamber arranged sequentially at intervals along the direction of the beam; wherein:
the carbide absorber is configured to adjust an energy range extracted by the proton accelerator according to tumor information of a target object;
the collimator is configured to calibrate the beam;
the first ionization chamber is configured to measure a real-time dose of the beam and feed the real-time dose back to a dose control system; and
the second ionization chamber is configured to measure an absolute dose of the beam and is located in front of the first window.

19. The FLASH therapy system according to claim 18, wherein the target object includes an animal or human suffering from a tumor.

20. The FLASH therapy system according to claim 17, further comprising a Faraday cup close to the second window, the Faraday cup being configured to measure a magnitude of the beam and collect a surplus beam.
